(19) United States
(12) Patent Application Publication
(10) Pub. No.: US 2024/0417324 A1
(43) Pub. Date: Dec. 19, 2024

(54) COMPOSITIONS AND METHODS OF DUST CONTROL

(71) Applicant: Biomason Inc., Durham, NC (US)

(72) Inventors: Ginger K. Dosier, Raleigh, NC (US); Victoria M. Durham, San Diego, CA (US); Thomas A. Hill, Dacula, GA (US); J. Michael Dosier, Raliegh, NC (US); Steven W. McAllister, Durham, NC (US)

(73) Assignee: Biomason Inc., Durham, NC (US)

(21) Appl. No.: 18/821,101

(22) Filed: Aug. 30, 2024

Related U.S. Application Data
(63) Continuation of application No. 18/641,275, filed on Apr. 19, 2024.
Application No. 18/641,275 is a continuation of application No. 17/570,312, filed on Jan. 6, 2022.
Application No. 17/570,312 is a continuation of application No. 16/781,622, filed on Feb. 4, 2020, now abandoned.
Application No. 16/781,622 is a continuation of application No. 15/066,692, filed on Mar. 10, 2016, now abandoned.
(60) Provisional application No. 62/200,288, filed on Aug. 3, 2015.
(60) Provisional application No. 62/188,556, filed on Jul. 3, 2015.
(60) Provisional application No. 62/130,854, filed on Mar. 10, 2015.

(51) Int. Cl. C04B 12/00; B05D 1/02; C04B 28/10; C04B 103/00; C04B 111/00; C04B 111/60; C12N 11/02; C12N 11/14

(52) U.S. Cl. CPC C04B 12/00; B05D 1/02; C04B 28/10; C12N 11/02; C12N 11/14; C12Y 305/01005; C04B 2103/0001; C04B 2103/0075; C04B 2111/00146; C04B 2111/60; Y02W 30/91

(57) ABSTRACT

Compositions and methods for producing materials for construction and for dust control utilizing enzyme producing cells, an amount of a nitrogen source such as urea, and an amount of calcium such as calcium chloride. Calcium contributes to the formation of calcium carbonate which creates a solid structure, layer or shield. One or more compositions containing components of the invention can be sprayed or otherwise applied to surfaces for erosion control, foundation support, prevention of sink hole formation, prevention of dust formation, or other applications. Ammonia, water and other by-products of the process can be recycled and reutilized for the same or other purposes including, for example, as fertilizers and energy sources, or independently fermented from selectively cultivated microorganisms.

### REFERENCE TO RELATED APPLICATIONS

This application is a continuation of U.S. application Ser. No. 18/641,275 filed Apr. 19, 2024, which is a continuation of U.S. application Ser. No. 17/570,312 filed Jan. 6, 2022, which is a continuation of U.S. application Ser. No. 16/781,622 filed Feb. 4, 2020, which is a continuation of U.S. application Ser. No. 15/066,692 filed Mar. 10, 2016, which claims priority to U.S. Provisional Application No. 62/200,288 filed Aug. 3, 2015, U.S. Provisional Application No. 62/188,556 filed Jul. 3, 2015, and U.S. Provisional Application No. 62/130,854 filed Mar. 10, 2015, the entirety of each of which is specifically incorporated by reference.

### FIELD OF THE INVENTION

The invention is directed to kits, compositions, tools and methods for dust control. More particularly, the invention is directed to materials and methods for dust suppression, with isolated enzymes, enzyme-producing bacteria or cells, or spores that give rise to enzyme-producing microorganisms.

### BACKGROUND

The built environment is primarily constructed using a limited palette of traditional materials: clay, concrete, glass, steel, and wood. Commonly used throughout history, masonry construction continues to make up a large part of the built environment, utilized for both load bearing structures and veneer construction. According to Chaisson, globally, traditional clay brick manufacturing produces over 1.23 trillion units per annum with a heavy dependency on non-renewable natural resources. Clay brick manufactured in coal-powered kilns emits approximately 1.3 pounds of carbon dioxide per unit. According to Burke, in total, brick manufacturing emits over 800 million tons of man-made CO₂ each year, and yet represents only one material currently used in building construction.

Fired clay bricks can be manufactured between 3-20 days, depending on the equipment and processes used. This range represents modem automated factories able to process bricks without manual labor, to the clamp method of bricks stacked around a burning fire used in many developing nations.

As an alternative to load bearing fired clay masonry, Concrete Masonry Units [CMU] are widely used as they are more economical, faster to manufacture and can serve as a structural typology for global construction. Comprised of concrete, these units are made with Portland cement, large aggregate and sand filler. According to Hanley of the United States Environmental Protection Agency, global carbon dioxide (CO₂) emissions from cement production were approximately 829 million metric tons of CO₂ in 2000.

These traditional materials contain a high-embodied energy, with components of concrete and steel mined from non-renewable resources. Approximately, forty-percent of global carbon dioxide is linked to the construction industry, primarily due to material production and disposal. Biologically grown materials can be pollution free and contain a low embodied energy, if produced as part of a local ecosystem.

Natural cement is created through chemical deposition and chemical processes associated with weathering, and can be found in various locations on the earth's crust. The formation of natural sandstones is primarily attributed to the precipitation of calcite cement. As an alternative to natural deposition, a form of natural cement has been produced with urease producing Sporosarcina Pasteurii, a nonpathogenic, common-soil bacterium has the ability to induce the production of calcite through a chemical reaction. The result is a hardened material formed in a process referred to by Stocks-Fischer as microbial induced calcite precipitation [MICP]. Applications include environmental stabilization of contaminated soils and encapsulation of hazardous and other contaminants in natural soils and acid mine tailings. Ramachandran and Jonkers have proposed the use of microbes to remediate cracks in concrete structures and the repair of cracks in monuments. According to DeJong and Whiffin, civil engineering researchers in the United States, Australia and the Netherlands have proposed the use of MICP for soil stabilization and erosion control.

A need exists for a process to manufacture building materials that does not impose the high energy costs associated with the manufacture of clay bricks and other conventional stone replacement, but utilizes readily available materials and is both economical and environmentally safe.

### SUMMARY

The present invention overcomes problems and disadvantages associated with current strategies and designs, and provides new tools, compositions, and methods for the manufacture of building materials.

One embodiment of the invention is directed to compositions comprising a support material to which is coupled urease-producing cells or cell spores and a transport medium and optionally a nutrient mix. Preferably the support material is organic or inorganic and comprises rock, glass (e.g. Poraver), wood, paper, metal, plastic, polymers, minerals or combinations thereof. Preferably the composition is a liquid, a gel, a sludge, a pump-able slurry, a dry powder or crystals and the support material is in the form of beads, grains, rods, strands, fibers, flakes, pulverized or crushed stone, crystals, fines, or combinations thereof. Preferably the support material is sand, glass, wood (e.g., residuals, pulp, sawdust, lignin), metal, polymers, fines (e.g., microcellulose), waste materials (e.g., ash, scrubber waste, residuals), co-cultured microorganisms or combinations thereof and the urease-producing cells or cell spores comprise yeast, algae, bacteria or eukaryotic cells, cell spores, anaerobic cells, or facultative anaerobic cells. Preferred bacteria are Sporosarcina pasteurii, Bacillus megaterium, Sporosarcina ureae, Proteus vulgaris, Bacillus sphaericus, Myxococcus xanthus, Proteus mirabilis, Helicobacter pylori, or variants, serotypes, mutations or combinations thereof, and preferred yeast, algae, bacteria or eukaryotic cells or cell spores are genetically engineered. The support material and the cells are preferably coupled via hydrophobic bonds, hydropyllic bonds, ionic bonds, nonionic bonds, covalent bonds, van der Waal forces, or a combination thereof and/or the support material is at least partially or totally encompassed by a film that promotes binding of the urease-producing cells. Preferred films comprise a polymer or a cell nutrient and preferably the composition contains a coloring agent which may be red, blue, green, yellow or any combination or shade thereof. Preferably the composition contains an identifying agent or a detectable marker such as a microscopic tag, a color, a nucleic acid or peptide, an enzyme or another substance.

Another embodiment of the invention is directed to kits for manufacturing solid forms comprising: the composition of the invention, a second composition containing nutrients for proliferation of the ureases-producing cells and/or germination of the cell spores; a plurality of sets of formworks wherein each set encloses the shape of at least one solid form and contains one or more porous panels; and a third composition comprising a calcium source (e.g., CaCl₂), a nitrogen source (e.g., urea) or both a calcium source and a nitrogen source. Preferably the kit is for the creation of solid forms such as, for example, rectangular, square, rounded, oval or an irregular shape. Preferred solid forms include but are not limited to blocks, boards, bricks, pavers, panels, tiles, or veneer. Preferably kits of the invention are for the manufacture of blocks such as, for example, concrete masonry, cinder blocks, foundation blocks, breeze blocks, hollow blocks, solid blocks, besser blocks, clinker blocks, high or low density blocks, or aerated blocks. Preferably the nutrients include amino acids, proteins, polysaccharides, fatty acids, vitamins and minerals.

Another embodiment of the invention is directed to methods for manufacture of solid forms comprising: mixing the composition of the invention with an aggregate material and water to form a mixture, wherein the aggregate material is largely composed of particulates of 5 mm or greater or particles of than 5 mm or less in diameter (e.g., fines); optionally apportioning the mixture into multiple form works wherein each form work contains at least one porous panel; adding a second composition to the mixture, wherein the second composition contains nutrients that promote proliferation of the urease-producing cells; adding a third composition to the mixture, wherein the third composition is a liquid that contains calcium; incubating the mixture for a period of time to form covalent bonds between the particulates; and removing the solid forms from the form works. Preferably the aggregate material comprises rock, glass, wood, paper, metal, plastic, polymers, minerals or combinations thereof, and/or mixing comprising spraying the composition as a liquid onto the aggregate material. Preferably the form works are substantially submerged during the incubating and air is bubbled to the submerged form works. Preferably a third composition is added to the mixture repeatedly during incubating which drains through the bottom panel and, optionally, is recycled. Preferably, incubating is performed under ambient conditions and the third composition contains calcium chloride, calcium acetate, calcium phosphate, calcium carbonate, calcium lactate, calcium nitrate, or a calcium salt. Preferably the pH of the mixture is monitored during the incubating. Preferably the solid forms are blocks, boards, bricks, thin bricks, pavers, panels, tiles, or veneer, stone (manufactured, cultured, colored), and the mixture further contains fibers or nanofibers that are, for example, fibers or nanofibers of wood, glass, plastic, metal or a polymer. Preferred fibers include, for example, polypropylene, HDPE, carbon fibers including high-strength carbon fibers, rayon, and biodegradable and non-biodegradable fibers such polymers of, for example, poly lactic acid, fibers of cellulose, minerals, chitin, lignin, and other plant materials. Preferably additional nutrients are added during incubating and the solid forms removed from the form works are dried.

Another embodiment of the invention comprises compositions containing urease producing cells or cell spores that are encapsulated or coated with nutrient media such as, for example, proteins or polysaccharides, or polymers such as poly lactic acid which is water soluble. Preferably the nutrient media further contains additional urease producing cells or cell spores.

Another embodiment of the invention comprises methods of dust control comprising: providing two compositions, wherein one composition contains an aqueous or dry mixture of viable cells or spores of urease-producing microorganisms and a transport media, and the other composition contains an aqueous form of calcium, wherein either or both compositions further contain urea and ingredients for growth of the microorganisms; and separately applying the two compositions to an area. Preferably the microorganisms comprise yeast, algae, bacteria, eukaryotic cells, or recombinantly engineered microorganisms, and also preferably the bacteria comprise Sporosarcina pasteurii, Bacillus megaterium, Sporosarcina ureae, Proteus vulgaris, Bacillus sphaericus, Myxococcus xanthus, Proteus mirabilis, Helicobacter pylori, or variants, serotypes, mutations, or combinations thereof. Preferably the transport medium maintains viability without promoting significant propagation of the microorganisms and may comprises minimal cell medium. Preferably the calcium comprises calcium chloride, calcium acetate, calcium phosphate, calcium carbonate, calcium lactate, calcium nitrate, and/or a calcium salt, and the ingredients for growth comprise one or more of water, nutrients, vitamins, minerals, amino acids, proteins, oils, fatty acids, saccharides and polysaccharides. Preferably methods of applying comprise spraying or misting and the area is a walking path, a mining area, tailings, gangue, a debris or waste pile, a cliff, a roadway, an aircraft landing area, a contaminated or hazardous area, or a construction site, or air space of a mining area, factory, manufacturing plant, or work area. Preferably, either or both compositions form a film on a surface that promotes binding of the microorganisms and may be aqueous. Also, the film may comprise a polymer, and either or both compositions may contain a coloring agent, or a detectable marker. Applying may comprise repeated applications of either or both compositions to the surface, and/or further applying another aqueous composition to the area.

Another embodiment of comprises kits for dust control comprising: a first composition containing a mixture of viable cells or spores of urease-producing microorganisms and a transport media; and a second composition containing a form of calcium, wherein either or both first and second compositions further contain urea and ingredients for growth of the mixture of viable cells or spores. Preferably the mixture of viable cells or spores comprise yeast, algae, bacteria, eukaryotic cells, or recombinantly engineered microorganisms, and preferably the bacteria comprise Sporosarcina pasteurii, Bacillus megaterium, Sporosarcina ureae, Proteus vulgaris, Bacillus sphaericus, Myxococcus xanthus, Proteus mirabilis, Helicobacter pylori, or variants, serotypes, mutations, or combinations thereof. Preferably the transport medium maintains viability without promoting significant propagation of the microorganisms, such as minimal cell medium. The form of calcium may comprise calcium chloride, calcium acetate, calcium phosphate, calcium carbonate, calcium lactate, calcium nitrate, and/or a calcium salt, and the ingredients for growth of microorganisms comprise one or more of water, nutrients, vitamins, minerals, amino acids, proteins, oils, fatty acids, saccharides and polysaccharides. Preferably either or both first and second compositions are aqueous, may further contain a polymer, a coloring agent, and/or a detectable marker. Kits may also comprise an apparatus for applying either or both first and second compositions.

Other embodiments and advantages of the invention are set forth in part in the description, which follows, and in part, may be obvious from this description, or may be learned from the practice of the invention.

### DETAILED DESCRIPTION

Traditional constructions materials such as clay bricks and concrete require enormous amounts of energy during the manufacture process. These processes are heavily reliant on burning natural resources such as oil, coal and wood. This reliance results in the consumption of massive amounts of energy resources and equally massive carbon dioxide emissions, thus a great dependency on limited energy sources. An alternative has been described which requires much less energy for manufacturing that utilizes enzymes produced by microbial cells. Typically, cells are aerobic and/or facultative anaerobic cells and include, for example, Sporosarcina pasteurii, Bacillus megaterium, Sporosarcina ureae, Proteus vulgaris, Bacillus sphaericus, Myxococcus xanthus, Proteus mirabilis, Helicobacter pylori and other strains, serotypes, variations, mutations and CRISPR modifications (clustered regularly interspaced short palindromic repeats). Cells produce the enzyme urease which, in the presence of calcium and nitrogen sources, forms calcite crystals. The process is generally referred to a microbial induced calcite precipitation (MICP), which can be performed with the cells or purified enzyme. As little to no heating is necessary, the energy savings in both expenses and efficiency is enormous.

The enzymes and/or the enzyme-producing cells are dispersed in a composition containing a nitrogen source and a calcium source, such as for example, urea, and calcium chloride with an aggregate material catalyzing the production of ammonia and carbon dioxide, increasing the pH level of the composition. The rise in pH forms a mineral precipitate combining calcium with carbon dioxide. The cells or other particles act as nucleation sites, attracting mineral ions from the calcium to the particle surfaces forming calcium carbonate crystals such as calcite crystals or other calcium carbonate polymorphs. The mineral growth fills gaps between the particles of aggregate, bio-cementing or bonding aggregate particles forming a solid. The resulting material exhibits a composition and physical properties similar to naturally formed sandstone, but whose hardness can be predetermined based at least on the structure of the initial components and the pore size desired.

Compositions and methods have been surprisingly discovered that are useful in the control of dust. The compositions include two solutions, a first solution containing a fluid plus urease enzyme or urease-producing cells or cell spores and a second aqueous solution containing calcium ions. The first or second solution may contain nutrients for the propagation of the cells or the spores, a nitrogen source such as, preferably urea, and a carrier such as clay or pond fines. Commercial sources of urease enzyme include, for example, jack beans. The enzymes can be maintained as a liquid, but are preferably lyophilized for ease of storage and transport, and rehydrated before use with a fluid such as, preferably, water, buffered water, or another hydrating agent that preserves enzyme activity. Preferable, pure enzyme is encapsulated in carbohydrate, lipid or other polymer microshells or spheres. Encapsulation techniques include, for example, encapsulation in nanoorganized microshells, and encapsulation in xanthan-alginate spheres. Preferred enzyme concentrations are from 0.5-5 mg/ml in 0.1 M phosphate buffer, pH 7.6. Preferably enzyme concentrations are from about 0.1 to 100 mg/ml, more preferably about 0.5 to 30 3.0 mg/ml, more preferably from about 0.5 to 2.0 mg/ml, and more preferably about 1.0 mg/ml. Enzyme can be further diluted prior to use to obtain a rate of 0.02-0.04 AA/minute. Enzyme activity can be measured by the reaction:

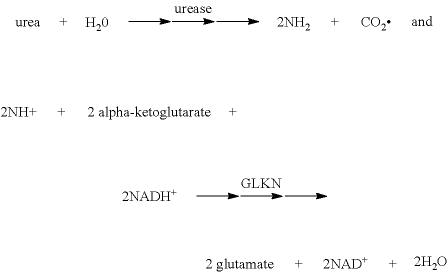

which couples ammonia production to a glutamate dehydrogenase reaction. Accordingly, one unit of enzyme results in the oxidation of one micromole of NADH per minute at 25° C. and pH 7.6.

This method for manufacturing construction materials through induced cementation exhibits low embodied energy and can occur at ambient pressures or higher or lower, and ambient temperatures or higher or lower. For example, preferred pressures are from about 10 psi to about 100 psi and all pressure values in between, also preferred at from about 14 psi to about 50 psi. Although higher pressures can be utilized, there is generally no need for the energy expense required. Preferred temperature ranges are from at least minus 20° C. to above 80° C., preferably from about 5° C. to about 50° C., preferably from about 15° C. to about 30° C., preferably from about 20° C. to about 25° C. Preferably, temperature ranges are below 30° C., below 40° C., below 50° C., below 60° C., or below 70° C. The ambient temperatures and conditions as well as the content of available aggregates can determine whether pure enzyme, lyophilized enzyme, spores, or live cells are utilized as the starting components. Living cells can be used in temperatures where mild weather conditions exist, whereas pure enzymes can be advantageous at more extreme conditions of cold or heat. Spores are used when immediate calcification is not required and the spores are provided sufficient time to germinate and express enzyme.

Processing also involves the production of quantities of by-products such as ammonia, not all of which is utilized in calcite formation. It is another embodiment of the invention to include an effluent recovery system in association with the production methodology. The recovery of ammonia from effluent converts the effluent to plain water which can be recycled or disposed of without any need for additional decontamination procedures. Preferred ammonia recovery methodologies include, for example, ion-exchange resins and commercially available processing such as Ammonia Electrolysis, zeolite, clinoptilolite and combinations thereof. Preferably, the ammonia recovered can be utilized in fertilizers, converted to nitrogen, utilized for energy generation or utilized for other applications.

One embodiment of the invention is directed to compositions comprising urease-producing cells or cell spores, urease enzymes (e.g., crude extract, or unpurified or purified enzymes) in a transport medium and optionally a nutrient medium. Transport medium includes, for example, growth media for urease-producing and/or other supporting cells, enzyme stabilizing media, reagent media, buffered solutions and combinations thereof. The composition may include or be combined with a support material which may be organic or inorganic and is preferably a solid or semi-solid and preferably contains holes or perforations and/or is otherwise porous. Organic support material includes, for example, biomass such as, preferably, moss, hay, straw, grass, sticks, leaves, algae, dirt, ash, dust, particulate material, refuse and combinations thereof. Inorganic material includes, for example, minerals, supplemental cementitious materials (SCM), pulverized or crushed rock, fines, and combinations thereof. Fibrous materials include sheets or tarps of burlap, paper, wood (e.g. residuals), cotton, or another natural or synthetic fiber. Non-natural and manufactured materials may also be used such as, for example, sheets of plastic, glass, fiberglass, vinyl, rubber, synthetic fabrics or combinations thereof. To the solid support is applied or otherwise introduced urease-producing cells, urease enzyme or simply other cells. Preferably these other cells would be useful to support the grown of the urease-producing cells or enhance the chemical processes involved and not otherwise interfere with the MICP process or to act as nucleation sites. Preferably these other cells are native or latent microorganisms in the local environment or provided with the mix, non-pathogenic, non-toxic and/or relatively harmless at the amount used, and easily obtained, present in the local environment or provided. Cells can be proliferated directly on the support material and, at a desired density or growth stage, the organic material evenly dispersed and/or thoroughly mixed into an aggregate material for manufacture of construction tools and products. Inorganic materials that can be used include, for example, rock (e.g., fines), sand, glass, wood, paper, metal, plastic, polymers, minerals, manufacturing or processing waste materials such as ash, carbon or wood residuals, any of which can be crushed or used whole or combinations thereof. Compositions may also be formed from waste materials that are otherwise hazardous (e.g., radioactive materials, materials with dangerous metal or poison content, contaminants from scrubbers, or other harmful materials) and formed into solid structures that can be stably stored or otherwise safely disposed.

Compositions of the invention may be sprayed or otherwise applied to sheets or mats or natural or non-natural materials and the sheets used, for example, to prevent erosion by formation of a calcium carbonate crust over a surface, a pile, a cliff or other structure that is subject to erosion. With the use of perforated or porous sheets or mats, the crust forms through the support material attaching the material on which the sheet has been placed. Nucleation sites for calcite formation can include, for example, polymers, fibers, fines, SCM, added Portland cement, powders, co-cultivated microorganisms, and combinations thereof. One or multiples layers of crusts can be formed on site. In this way, erosion and dust control can be substantially reduced or eliminated in defined areas. Importantly, in this fashion the sheet can be easily replaced over time and/or a fresh composition of the invention re-applied to the surface as needed or as desired. Mats provide the additional benefit of “seeding” the site for rehabilitation once operations have ceased-thereby allowing site recovery efforts to proceed such as returning the site to a natural state. This is especially applied to mining sites in which mining operations have ceased. Preferred areas for application include a walking path, a mining area, tailings, gangue, a debris or waste pile, a cliff, a roadway, an aircraft landing area, a contaminated or hazardous area, or a construction site, or air space of a mining area, factory, manufacturing plant, or work area.

In a preferred embodiment of the invention, compositions of the invention are applied to a surface area as a liquid, a gel, a slurry, a sludge, a semisolid or a dry powder. Spores and/or microorganisms of a composition of the invention produces enzymes that catalyze formation of a crust of calcium carbonate in the presence of liquid, which is preferably water, buffered water or another aqueous material. A nutrient mix suitable for the particular microorganism can be included with the cells. When the composition dries, the crust remains and cells go dormant. As cells self-propagate, provided sufficient nutrients and/or substrate materials are present, new crust will form whenever sufficient aqueous liquid is provided. In a preferred embodiment, nutrients and/or substrate materials may be distributed over the surface area of interest in slow-release or timed-release form such as dry components with a pre-determined rate of dissolution. Reformation may simply be a matter of re-applying water that dissolves nutrient and/or substrate thereby re-activating the microorganisms. The re-activated microorganisms produce enzyme that forms crust. This process can be repeated with or without the reapplication of microorganisms, nutrients and/or substrates, or with only occasional additions. This process may be coupled with weather events so that the rain provides the source of water. By providing a composition that provides microorganisms and/or spores and contains slow-release nutrients and/or substrate, crust can be reformed over an area repeatedly over long periods of time.

Preferably compositions of the invention including all necessary components such as microorganism, spores and/or enzymes, a nitrogen source, and a calcium source and optionally nucleation sites (e.g., powders, fines, co-cultured microorganisms and/or other materials), are applied to a surface, such as, for example, a dirt road, or a structure such as a hill or cliff. Microorganisms proliferate and produce enzyme which h catalyzes the formation of a crust of calcium carbonate over the road surface. As vehicles travel over the road, the crust breaks eventually turning the crust into dust. Periodically the geographic region experiences rain or other forms of precipitation that dissolves slow-release nutrients and/or substrate materials, thereby promoting proliferation of dormant microorganisms. The microorganisms produce enzymes which catalyze new crust formation over the road. In periods of reduced precipitation, an aqueous solution is re-applied to the road to activate the microorganisms which may or may not contain additional nutrients and/or substrate materials. Preferably the composition is a liquid, a gel, a slurry, a sludge or dry powder and the support material may be in the form of beads, grains, rods, strands, fibers, flakes, dirt, biomass, sand, pulverized or crushed stone, fines, supplemental cementitious materials (SCM), crystals, co-cultivated microorganisms, or combinations thereof. Fines sizes are preferably equal to or less than 250 micron, more preferably equal to or less than 200 micron, more preferably equal to or less than 150 micron, or more preferably equal to or less than 100 micron (reference examples include micron size of beach sand=700, micron size of fine sand=250; micron size of Portland cement=74; micron size of silt=44; micron size of smoke=2). Support material and aggregate material can be the same or different. Preferably the support or aggregate material is sand, glass, metal, added Portland cement, SCM, fines, co-cultivated microorganisms (e.g., native, latent, local, added, or genetically modified microorganisms), or combinations thereof and the urease-producing cells or cell spores comprise yeast, algae, anaerobic cells, facultative anaerobic cells, bacteria or eukaryotic cells or cell spores. Preferred bacteria are Sporosarcina pasteurii, Sporosarcina ureae, Proteus vulgaris, Bacillus sphaericus, Myxococcus xanthus, Proteus mirabilis, Helicobacter pylori, or variants, serotypes, mutations or combinations thereof, and preferred yeast, algae, bacteria or eukaryotic cells or cell spores are genetically engineered. Other enzyme producing bacteria that are capable of biocementation include Sporosarcina ureae, Proteus vulgaris, Bacillus sphaericus, Myxococcus xanthus, Proteus mirabilis and Helicobacter pylori, although proper concerns should be given to pathogenic strains. Combinations of any of these strains as well as functional variants, mutations and genetically modified stains may be used as well. The support material and the cells are preferably coupled via hydrophobic bonds, hydropyllic bonds, ionic bonds, non-ionic bonds, covalent bonds, van der Waal forces, or a combination thereof and/or the support material is at least partially or totally encompassed by a film that promotes binding of the urease-producing cells. Preferred films comprise a polymer or a cell nutrient and preferably the composition contains a coloring agent which may be red, blue, green, yellow or any combination or shade thereof. Preferably the composition contains an identifying agent or a detectable marker such as a microscopic tag, a color, an enzyme or another substance.

Support materials and/or aggregate materials may contain additional components that provide an advantage to the construction materials. For example, chemicals and/or additional cells (e.g., native, local or latent bacteria, yeast, eukaryotic cell, algae, and recombinant variations thereof), can be included that produce enzymes, cofactors and/or other chemicals useful in breaking down stains in and/or acquired by the final product and/or additional nucleation sites. Stains include stains from air pollution, soot, mold or animal waste products. Alternatively, the chemical or enzymes may impart color, texture or a desired function or appearance to the final product.

Another embodiment of the invention is directed to kits for manufacturing solid forms comprising: the composition of the invention, a second composition containing nutrients for proliferation of the ureases-producing cells and/or germination of the cell spores; a plurality of sets of formworks wherein each set encloses the shape of at least one solid form and contains one or more porous panels; and a third composition comprising a calcium source (e.g., CaCl₂), a nitrogen source (e.g., urea) or both a calcium source and a nitrogen source. Preferably the kit is for the creation of solid forms such as, for example, rectangular, square, rounded, oval or an irregular shape. Preferred solid forms include but are not limited to blocks, boards, bricks, pavers, panels, tiles, counter tops, or veneer. Preferably kits of the invention are for the manufacture of blocks such as, for example, concrete masonry, cinder blocks, foundation blocks, breeze blocks, hollow blocks, solid blocks, besser blocks, clinker blocks, high or low density blocks, or aerated blocks, thin bricks, manufactured stone, cultured or colored stone. Nutrient compositions of the invention may contain nutrient media to maintain and/or allow the cells to flourish and proliferate. The various types of nutrient media for cells, and in particular, bacterial cells of the invention are known and commercially available and include at least minimal media (or transport media) typically used for transport to maintain viability without propagation, and yeast extract, molasses, and corn steep liquor, typically used for growth and propagation. Preferably the nutrients include amino acids, proteins, polysaccharides, fatty acids, vitamins and minerals.

Another embodiment of the invention is directed to methods for manufacture of solid forms comprising: mixing the composition of the invention with an aggregate material and water to form a mixture, wherein the aggregate material is largely composed of particulates of less than 5 mm in diameter (e.g. less than or about 4 mm, less than or about 3 mm, less than or about 2 mm, or less than or about 1 mm); apportioning the mixture into multiple form works wherein each form work contains at least one porous panel; adding a second composition to the mixture, wherein the second composition contains nutrients that promote proliferation of the urease-producing cells; adding a third composition to the mixture, wherein the third composition is a liquid, powder or paste that contains calcium; incubating the mixture for a period of time to form covalent bonds between the particulates; and removing the solid forms from the form works.

Alternatively, the compositions may be combined and added together to the material within the form works or combined with the material prior to addition to the form works. Another embodiment of the invention is directed to the structure and composition of form works. Preferred form works comprises a thermoplastic material that can be molded or extruded into a desired shape. Preferred thermoplastics include, but are not limited to plastics such as polypropylene, polystyrene, polyethylene including HDPE (high density polyethylene), LPDE and reclaimed LDPE (low density polyethylene), and cross-linked polyethylene, glass and most any formable polymer. Preferably, the polymer material is provided as pellets or lens shapes that range in thickness and uniformity. The pellets are filled in a porous mold and steamed under pressure (the mold is not under pressure, pressure just from the steam). The resulting product provides a designed flow directional material, and changes to the gradation impact the flow direction, speed and retained saturation.

Another embodiment of the invention is directed to compositions and structures that do not require formworks (e.g. frameless manufacturing) wherein structures are formed from a combination of the components of the invention plus polymers and/or thermoplastics that are compressed with a compaction device and retain the desired structure. Preferred compression devices include hydraulic presses and preferred pressures are 100 psi or greater, 250 psi or greater, 500 psi or greater, 1000 psi or greater, 2000 psi or greater, 3000 psi or greater, 4000 psi or greater, 5000 psi or greater, Preferred components of the invention include all the components to form calcium carbonate structures in the form of a sludge or paste. The compaction device compresses the components with added pressure into a form that is maintained and dries without significant alterations of the resulting form. Preferred polymers and thermoplastics include, but are not limited to plastics such as polypropylene, polystyrene, polyethylene including HDPE (high density polyethylene), LPDE and reclaimed LDPE (low density polyethylene), and crosslinked polyethylene, glass, carbohydrates such as starches, lignin, and most any formable polymer. Compressed form can be generated rapidly from a think slurry or sludge and maintains its shape during calcite formation. Preferably calcite formation is accomplished in vapor chambers (e.g., at greater than ambient pressures) that contain increased vapor pressures or are sprayed or misted, wherein the vapor, mist or spray preferably comprises nutrients or chemical substrates. Preferred forms include blocks, bricks, thin bricks, manufactured or cultured stone, pavers, or any useful structure.

Preferably the multiple form works or compression devices create 5, 10, 50, 100, 500, 1,000, 10,000, 100,000 1,000,000 or more forms at a time. The number of form works or compression devices that can be simultaneously utilized is limited only by the complexity of the mechanics and space available. These form works or devices may be stacked or provided in a single layer or pallet. Formwork may have vertical walls which are connected together forming cavity there between to receive the aggregate material. Formworks may also have a floor and, alternatively, the bottom of the formwork may be left open if supported by a porous surface such as soil, or aggregate and composition may be mixed and pressed into molds or extruded. Preferably, vertical walls are at least the inside surfaces thereof, are made of a non-reactive, non-porous material or coating such as cast or extruded acrylic resin. This enables one to easily remove the construction material or the brick from the formwork after it has solidified. In addition, the vertical walls and floor of formwork or pressure devices may have designs that form surface textures in the resulting bricks or other structures (e.g., lines, circles, waves, groves, sketches, images, etc.).

Preferably the aggregate material comprises rock, glass, fiberglass, wood (residuals, pulp, sawdust, lignin), biomass, paper, metal, plastic, polymers, rubber, imitation rubber, vinyl, minerals, co-cultured microorganisms, waste materials (e.g., ash, carbon, scrubber waste, radioactive pellets) or combinations thereof, and/or mixing comprising spraying the composition as a liquid onto the aggregate material. Preferably the form works are substantially submerged during the incubating and air is bubbled to the submerged form works. Preferably a third composition is added to the mixture repeatedly during incubating which drains through the bottom panel and, optionally, is recycled. Preferably, incubating is performed under ambient conditions and the third composition contains calcium chloride, calcium acetate, calcium phosphate, calcium carbonate, calcium lactate, calcium nitrate, or a calcium salt. Preferably the pH of the mixture is monitored during the incubating. Preferably the solid forms are blocks, boards, bricks, pavers, panels, tiles, or veneer, and the mixture further contains fibers or nanofibers that are, for example, fibers or nanofibers of wood, glass, plastic, metal or a polymer. The solid forms can be partially or uniformly porous containing a network of holes or gaps. Holes can be of a predetermined size and/or structure such as, for example, at least 5 microns, at least 10 microns, at least 20 microns, or at least 50 microns in diameter. Alternatively, solid forms can be manufactured with materials that provide virtually no or few holes. For example, adding a non-porous material to the aggregate mixture can create complex and extended pathways that render the form impermeable to fluids.

Another embodiment of the invention comprises compositions containing urease producing cells or cell spores that are coated with nutrient media. Preferably the nutrient media further contains additional urease producing cells or cell spores, and/or nutrients to promote the proliferation of additional cells that have been added to the aggregate that are beneficial to the final product.

Another embodiment of the invention is directed to compositions, methods and systems for the treatment of aggregate materials comprised of particles with a composition comprising one or more of a nitrogen source such as for example urea, a calcium source (e.g., calcium ions) and urease or urease producing cells. Preferably particles have a diameter (e.g., actual, average or effective diameter) of about 50 mm or less, preferably about 25 mm or less, preferably about 20 mm or less, preferably about 10 mm or less, and preferably about 5 mm or less. In one preferred embodiment, aggregate material can also be about 1 mm or less and preferably about 0.5 mm or less, more preferably about 0.1 mm or less, and more preferably about 50 μm or less. Especially preferred particles sizes include from about 10 μm to about 1 mm, from about 100 μm to about 0.5 mm, from about 200 μm to about 1 mm, from about 1 μm to about 200 μm, from about 10 nm to about 1 μm, and from about 10 nm to about 40 nm, and various combinations 15 thereof. Particles include, for example, spores, carbon dust, dust or soot from cement or brick manufacture, cement block manufacture, foundry operations, grinding limestone, sand tailings, mining, smelters, paint manufacturing and byproducts of other manufacturing processes such as slag. Particles may be obtained and collected from available or implemented dust control procedures. Particles may be of mixed sizes including but not limited to sizes equal to and greater than preferred sizes, particles equal to and less that preferred sizes, and combinations of preferred sizes and mixtures thereof. Particles that are aggregates and more sizable particles may include recycled and/or recyclable materials. The nitrogen source of the composition may be a single chemical, such as urea of any grade and purity and is preferably commercially obtained. Calcium ions are preferably obtained from commercially available sources such as, for example, calcium chloride. Urease enzyme or urease-producing bacteria may be included in the composition. Urease-producing bacteria include, but are not limited to the bacteria Sporosarcina pasteurii, Sporosarcina ureae, Proteus vulgaris, Bacillus sphaericus, Myxococcus xanthus, Proteus mirabilis, Helicobacter pylori and combinations thereof. Urease producing cells includes non-viable cells that contain enzyme such as, for example, mycells, cells composed of lipids or fatty acids, and cells containing urease. Urease and/or urease-producing cells may produce or release a predetermined amount of enzyme over a defined period of time. Preferably, the amount of urease released per cell is sufficiently rapid to allow for the rapid creation of calcium carbonate in the presence of nitrogen and calcium ions.

Preferably, particles are combined with a nitrogen source (e.g., urea), urease and/or urease producing cells, calcium ions and preferably water to create a homogenous slurry. The slurry can be painted or sprayed onto objects and/or surfaces creating a layer or crust, molded into forms that solidify into objects which may be complete or partially solid, or otherwise pooled for immersion or dipping of objects to be coated with the slurry material again creating layers or a crust over the object surfaces. Objects may contain one or more layers as desired, and layers may be permeable or impermeable to water or improve resistant to wear from weather conditions such as sun damage, snow, ice and rain. Slurries that provide increased resistance are preferably composed with aggregate materials that are particles of less than 0.1 mm diameter. As the liquid dries, calcium carbonate bonds form between the particles and/or the particles and the object. The result can be an object containing an outer shell of hardened calcium carbonite or a formed structure. Objects that can be manufactured according to the invention and/or layered with a crust or coating of the invention include, but are not limited to bricks, cement blocks, pavers, counter tops, glass, fiberglass, polymer and acrylic structures, siding, walls, yard art, slate and rock structures, tiles, paving stones, steps, roofing material, gutters, cement walls and planks, patios, balconies, fencing and combinations thereof.

Another preferred embodiment of the invention comprises producing ammonia and/or other compounds (e.g., ammonia, organic acids, alcohols, phenolics, sulfides) by fermentation of microorganisms (e.g., microorganisms that ammonia monooxygenase, hydroxylamine oxidoreductase, nitrifying bacteria). Preferably microorganisms are selectively cultured to maximize generation of the desired enzymes. Hyper-ammonia producing microorganisms include, for example, ruminant-derived microorganisms, intestinal microorganisms, Peptostreptococcus sp., Clostridium sp., Calliandra sp., Atopobium sp., Desulfomonas sp., and the like. Isolated ammonia can be recycled or utilized in other processes such as in fertilizers and energy production.

Another embodiment of the invention comprises spraying the slurry of the invention onto a natural geological or man-made surface such as a cliff, a dune, an aggregate pile, a ledge, a supporting wall, ores, a foundation, minings, tailings, piles of waste materials from a manufacturing process, construction site, or another structure for which additional support or structuring is desired. Such support is advantageous in convenience and financial considerations as compared to providing additional support of the structure of interest with convention building systems. In addition, and preferably, slurry of the invention can be provided to geological surfaces such as soil around buildings to provide building support, erosion suppression, prevention and/or repair of sink holes, or to create foundation structures to provide solid support and/or stabilization of buildings and other structures, and combinations thereof.

Another embodiment of the invention is directed to compositions, methods and systems comprising a slurry of one or more of water, a nitrogen source (e.g., urea), optionally a calcium source (e.g., CaCl₂), and urease or urease-producing cells, but without the addition or any aggregate material such as, for example, without sand, soil, dust, silt or other particles as aggregate materials. Preferably the slurry contains at least water, a nitrogen source (e.g., urea), optionally a calcium source (e.g. CaCl₂), and urease or urease-producing microbes, which may include microbial nutrients as appropriate. Preferably the calcium source provided is another composition and the slurry is calcium-free. This liquid slurry is sprayed, painted, or otherwise placed directly on or in an aggregate material, or formed in a mold of most any shape or structure containing aggregate material, optionally followed by application of the calcium composition. The combination of the aggregate and the aforementioned slurry forms a solidified object, covering or layer (or multiple layers), such as, for example, as a building foundation, a molded object, a layer covering an object, or another desired form. One of the advantages of this technique is that aggregate material does not need to be shipped and there is a concomitant associated savings. Preferably aggregate material is immediately available on site or locally available within an acceptable distance. The addition of the slurry to the already-present aggregate creates a solid or more hardened form of a structure efficiently in situations where transporting or otherwise moving aggregate materials would be difficult, inefficient or impractical such as but are not limited to situations involving creation of, repair of or to further support building foundations and other repairs.

Another embodiment of the invention is directed to compositions and method comprising a slurry of the invention combined with an aggregate material, further containing multiple solid structures that are either hollow or otherwise of lighter weight than the aggregate material. The resulting structure containing the additional objects produce solid objects of lighter weight than objects made of only aggregate material and slurry. Alternatively, it may be desirable to increase the weight of the object by adding objects that are heavier than the aggregate material. Such heavier objects include, but are not limited to rebar or remesh, metal forms, strengthening material and other heavier materials. These additional objects include, but are not limited to plastic, wood, steel, metal, polymer, rods, balls geometric structures, which may be solid, perforated or hollow. Alternatively, the additional objects may be included that have aesthetic properties such as, for example, predetermined colors, materials, functions, properties and designs. This is advantageous when light weight objects are desired, wherein the structure retain sufficient strength for the intended purpose such as, for example, a specific desired compression strength, tensile strength, yield strength, ultimate strength, Young's modulus, elastic modulus, elastic strength, stiffness, hardness, toughness, stress resistance, and combinations thereof.

Another embodiment of the invention is directed to compositions, methods and systems comprising a variety of substrates combined with a slurry of the invention. The addition of sand, fines, silt, or dust (which are lighter and have smaller particles than soil or other aggregates) to urea, urease, optionally calcium and water create lighter structures with equivalent or nearly equivalent support strengths. Preferably the slurry is largely calcium free and a calcium-containing composition is provided in a second application. Advantages of lighter structures include a lower cost of production and a higher efficiency of production, as well as other benefits such as efficiency or manufacture and formation of structures. Preferably, urease enzymes are used to increase the solidification of the structure as compared to the use of enzyme-producing cells. In addition, enzymes are smaller in molecular structure than cells and will pass through smaller pore sizes of aggregate materials having small pore sizes. Also in addition, one or more chemical or compounds can be included to increase the density and/or weight of the liquid composition so that compositions settle quickly or are sufficiently sticky to a surface (e.g., as a gel, foam or semi-solid).

Another preferred embodiment of the invention comprises composition, systems and methods for forming solid or porous solid structures according to the invention that are lighter in weight as compared to convention structures composed of clay or cement. Preferably, the invention comprises creating a spatial gap within the solid structure during manufacture as the structure hardens. This gap can be in the form of holes, tubes, bubbles, or any other three-dimensional shape. A pre-formed shape made of the same aggregate material or materials, or of a different, preferably lighter material can be immersed into the wet, un-hardened slurry of the invention either with or without aggregate material. When the slurry fully formed around that desired shape, the resulting object will weigh less than conventionally prepared objects, such as, for example, clay bricks, cement blocks, pavers, stone composites, or another solid structure composed of one or more aggregate materials. The resulting solid object has an increased strength, new or enhanced aesthetic or performance characteristic, additive or a combination thereof.

Another preferred embodiment of the invention comprises composition, systems and methods for forming protective layers or coverings to solid structures. Preferably the slurry of the invention fills and closes pores in the solid structure (e.g., a fabric impregnated with one or more of microorganisms, nutrients, substrate materials, nucleation sites) so as to provide effective barriers to liquids (e.g. water), gasses (e.g. pollution) or other substances that may impregnate or contaminate a solid structure. Such compositions can be used for erosion control and structural support.

Another embodiment of the invention comprises compositions, systems and method for dust control of, for example, walking paths, piles, aircraft runway, taxi ways, and parking areas, cliffs, vehicle roadways and other large surfaces. Slurries of the invention can be substituted for oils and other dust control compositions presently used on dirt, gravel and other road surfaces to minimize the amount of dust created from vehicles. Slurries of the invention can be sprayed or vaporized from trucks or other vehicles as a liquid, or administered (e.g., spraying) as a dry composition to be activated when wetted, onto surfaces forming a hardened crust to the road or other surface. Slurries comprising substrates and living urease-producing microbes plus nutrients cover road surfaces with a self-renewable crust. An initial application can include microorganisms and optionally included with subsequent applications which may only contain substrate materials. As vehicles pass over the road, the crust may be damaged from the weight of the vehicle, but a crust is recreated and repaired by the presence of the living-slurry. Preferably slurry of the invention for dust control contains no aggregate or only aggregate of 0.5 mm or less in diameter.

Another embodiment of the invention comprises adding slurry of the invention, either with or without aggregate, to conventional procedures for the manufacture of construction materials such as, for example, clay brick, cement blocks, pavers, and other substances. Slurry additions can be included as desired at from 0.0001 percent to 99 percent of the dry weight of the resulting product or empirically determined from the type of aggregate used. Preferably the slurry addition by dry weight is from 1 to 50 percent, from 2 to 75 percent, from 30 to 60 percent, from 25 to 80 percent, from 10 to 25 percent or any combination there.

Another embodiment of the invention comprises creation of a slurry of the invention with which will solidify at a predetermined time. Preferably, slurries contain a predetermined amount of nitrogen and calcium sources as substrates and a predetermined amount of enzyme that solidifies within a desired time frame. Solidification conditions may include the temperature of use, which can be included in the calculations to determine solidification times preferably experimentally or empirically.

The following examples illustrate embodiments of the invention, but should not be viewed as limiting the scope of the invention.

### EXAMPLES

### Example 1

Reducing dust of surface mining sites is required by MSHA (mining version of OSHA) regulations. Current methods used in the industry include the use of various polymers or chemicals, with the most common being the continuous spray application of water, oils and other dust control liquids. The objective for surface mine dust control is to make fine dust (a byproduct of aggregate mining) heavier than air to prevent respiratory and visibility hazards. According to the invention, micro-organisms are applied either with a nutrient and/or transport material or in association with any conventional treatment for such dust control and/or surface cementation and include production of a calcite cement (CaCO₃) in combination with urea (nitrogen/carbon) and a calcium source. Cells and/or nutrient materials are included with an initial application, and optionally with subsequent or follow-on applications. Preferably, applications are of light-weight materials that are quick to cement using the same strains of urease-producing bacteria as used in the formation of bricks, pavers and other solid forms. Alternatively, cyanobacteria, a photosynthetic microorganism that fixes nitrogen from the atmosphere, is substituted for or used in addition to urease-producing bacteria, which reduces nutrient input needs.

### Example 2

Recovery systems seek to address: (a) returning effluent to a viable state to be re-used as influent (water becomes a capital expense rather than a consumable material), and (b) the extraction of commercially valuable byproducts from the effluent stream. Preferably, the bio-cementation process of the invention is useful for the primary production of by-products as products such as, for example, using urease producing microorganisms for the manufacture of ammonia/ammonium and/or free calcite. By-products are excess material to be reduced through optimization and/or accounted for in influent formulations. Ammonia as a recoverable byproduct has commercial value in both fertilizer and alternative fuel applications.

There are at least two ammonia extraction methods. First, granular zeolite cliniptilolite mineral aggregate is used as an air filter for extracting ammonia gas, and as a liquid filter for extracting ammonium from effluent. Ammonia-saturated zeolite has potential applications as a fertilizer, fertilizer additive, and/or fertilizer component. Second, an electrode based system is used for conversion of aqueous ammonia/ammonium as a hydrogen fuel source for electricity production.

Effluent, further treated or not, is a fuel source for other ammonia-based energy production technologies and recycling technologies including recycling of water, calcite and byproducts.

Settling tanks, mesh filters, fabrics and/or hydrocyclones are used for the removal of free calcite in solution wherein, and preferably, the micro-organisms remain. This material is an inoculation source for new biocement formation and fertilizer applications (calcium available for plant cell wall formation, and microbes available for soil denitrification).

### Example 3

Biologically-formed Microcrystalline Calcium Carbonate was produced using a urease-producing microorganism (S. pasteurii) grown in a liquid fermentation medium containing urea. The media was agitated to create a uniform suspension. At late stage growth of the culture, calcium ions were added in the form of a calcium chloride solution to a saturation of molar equivalency with the urea. Urease activity results in the hydrolysis of urea (2NH₂CO) into ammonium (NH₄) and carbon (C). Carbon combines with calcium (Ca) to produce calcium carbonate (CaCO₃). Calcium carbonate crystals formed ranged in size from 50 μm to 0.1 μm and generally “regular” (e.g., spherical) in shape. Calcium carbonate was separated from the solution using one or more of centrifugation, settling tanks, hydro-cyclones, or decanting. The method was performed as a batch process and also as a continuous production line.

A variation of this method was used to increase particle size by using fine aggregate materials to create an agglomerate, which also improved liquid-solid separation. In this variation, fine aggregate with a Mesh Scale of 70 micron size was added to the solution during the fermentation process and at a quantity that did not exceed the ability for the agitation to keep the fine aggregates in suspension. Following the addition of calcium ions, the calcite bonds to, and bonds together the fine aggregate, creating larger, heavier particles.

### Example 4

The method of Example 3 is performed with co-culture of a second organism, Delaya venusta. The co-culture process is developed for a single fermentation within a single reactor, alternating fermentations within a single reactor, or by circulating media between two separated fermentations in separate reactors, wherein the reactor is either a liquid state reactor (e.g., batch, batch-fed, or continuous), or a solid-state reactor such as an aggregate-unit (e.g., bricks).

### Example 5

In another example, a first composition is prepared containing an aqueous mixture of viable cells or spores of urease-producing microorganisms suspended within a transport media that promotes viability and not growth and/or germination of spores, but with minimal or no calcium. A second composition is prepared that contains an aqueous form of calcium, calcium chloride. A third composition is prepared containing urea and ingredients for growth of the microorganisms or, alternatively, the urea is added to the first composition and the ingredients are added to the second composition. The compositions are than applied to an area simultaneously or sequentially and, if sequentially, in any order. To an area such as a mining operation, dust in the atmosphere is significantly reduced and mostly eliminated.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the following claims.

What is claimed is:

1. A method for manufacturing a construction material, comprising:
providing an aggregate mixture that includes a plurality of aggregate particles, urease-producing cells or cell spores thereof, a first composition comprising nutrients, and a second composition comprising a calcium source and a nitrogen source; and
incubating the aggregate mixture for a period of time to form calcium carbonate bonds between at least a subset of the plurality of aggregate particles, the calcium carbonate bonds are formed from biological activity of the urease-producing cells at a pressure that is greater than ambient pressure.

2. The method of claim 1, wherein:
the incubating the aggregate mixture includes incubating the aggregate mixture while the aggregate mixture is within a formwork that includes one or more porous panels.

3. The method of claim 2, wherein:
the one or more porous panels include a perforated panel or a bottom panel.

4. The method of claim 1, wherein:
the incubating the aggregate mixture includes incubating the aggregate mixture while the aggregate mixture is within a porous mold.

5. The method of claim 1, wherein:
the incubating the aggregate mixture includes incubating the aggregate mixture while the aggregate mixture is within a compaction device.

6. The method of claim 1, wherein:
the incubating the aggregate mixture includes incubating the aggregate mixture while providing a fluid that includes one of the calcium source, the nitrogen source, or the nutrients to the aggregate mixture.

7. The method of claim 6, wherein:
the aggregate mixture is within a formwork that includes one or more porous panels and the fluid drains through the one or more porous panels while incubating the aggregate mixture.

8. The method of claim 1, further comprising:
adding a suspension or pump-able slurry of the urea-producing cells or cell spores thereof in water to the aggregate mixture while incubating the aggregate mixture.

9. The method of claim 1, wherein:
the incubating the aggregate mixture includes pumping a slurry that includes the urea-producing cells or cell spores thereof into the aggregate mixture.

10. The method of claim 1, wherein:
a urease concentration during incubation of the aggregate mixture is between 0.1 and 100 mg/ml.

11. The method of claim 1, wherein:
the calcium carbonate bonds are formed at a pressure that is greater than ambient pressure and less than 100 psi.

12. The method of claim 1, wherein:
the construction material comprises a block, brick, paver, panel, tile, or counter top; and
the aggregate mixture further comprises fibers or nanofibers.

13. The method of claim 12, wherein:
the fibers or nanofibers are of wood, glass, plastic, mineral, metal, or a polymer.

14. The method of claim 13, wherein:
the polymer consists of polypropylene, HDPE, carbon fibers, rayon, poly lactic acid, cellulose, chitin, or lignin.

15. The method of claim 13, wherein:
the calcium source comprises calcium chloride, calcium acetate, calcium phosphate, calcium carbonate, calcium lactate, or calcium nitrate; and
the nitrogen source is urea.

16. A method for manufacturing a construction material, comprising:
providing an aggregate mixture that includes a plurality of aggregate particles, urease-producing cells, one or more nutrients, a calcium source, and a nitrogen source; and
incubating the aggregate mixture for a period of time to form calcium carbonate bonds between at least a subset of the plurality of aggregate particles, the calcium carbonate bonds are formed from biological activity of the urease-producing cells at a pressure that is less than ambient pressure.

17. The method of claim 16, wherein:
the calcium carbonate bonds are formed at a pressure that is less than ambient pressure and greater than 10 psi.

18. The method of claim 16, wherein:
the incubating the aggregate mixture includes incubating the aggregate mixture while the aggregate mixture is within a formwork that includes one or more porous panels.

19. A method for manufacture of solid forms, comprising:
apportioning an aggregate mixture into one or more formworks wherein each formwork contains at least one porous panel, wherein the aggregate mixture comprises a composition comprising urease-producing cells and/or cell spores thereof, mixed with an aggregate material and water, and wherein the aggregate material comprises particulates of less than 50 mm in diameter;
providing a second composition to the aggregate mixture, wherein the second composition contains a calcium source and/or a nitrogen source; and
flowing the second composition through the aggregate mixture for a period of time to form calcite bonds between the particulates.

20. The method of claim 19, wherein:
the composition includes the spores of the urease-producing cells and a transport medium, the transport medium includes growth media for the urease-producing cells.
